(19) United States
(12) Patent Application Publication
(10) Pub. No.: US 2010/0256417 A1
(43) Pub. Date: Oct. 7, 2010

(54) PROCESS FOR THE PRODUCTION OF AROMATIC AMINDES IN THE PRESENCE OF A PALLADIUM COMPLEX COMPRISING A FERROCENYL BIPHOSPHINE LIGAND

(75) Inventors: Harald Walter, Basel (CH); Ulrike Nettekoven, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/376,583

(22) PCT Filed: Aug. 6, 2007
(86) PCT No.: PCT/EP07/06934

§ 371 Date: May 11, 2010

(30) Foreign Application Priority Data
Aug. 6, 2006 (EP) 06016478.7
Sep. 21, 2006 (EP) 06019748.0

(51) Int. Cl. C07C 209/10

(52) U.S. Cl. 564/307; 564/407

(57) ABSTRACT

The present invention relates to a process for the preparation of compounds of Formula (I), wherein R₁ is 1,3-dimethyl-butyl, 1,3,3-trimethyl-butyl or a group A₁, wherein R₃, R₄ and R₅ are each independently of the others hydrogen or C₁-C₄alkyl; and R₂ is hydrogen; or R₁ and R₂ together from the group A₂, wherein R₆, and R7 are each independently of the other hydrogen or C₁-C₄alkyl; or R₁ and R₂ together from the group A₃, wherein R₅ and R₉ are each independently of the other hydrogen or C₁-C₄alkyl; wherein a compound of Formula (II) wherein R₁ and R₂ are as defined for formula I and X is bromine or chlorine, is reacted with ammonia in the presence of a base and a catalytic amount of at least one palladium complex compound, wherein the palladium complex compound comprises at least one ferrocenyl-biphosphine ligand.

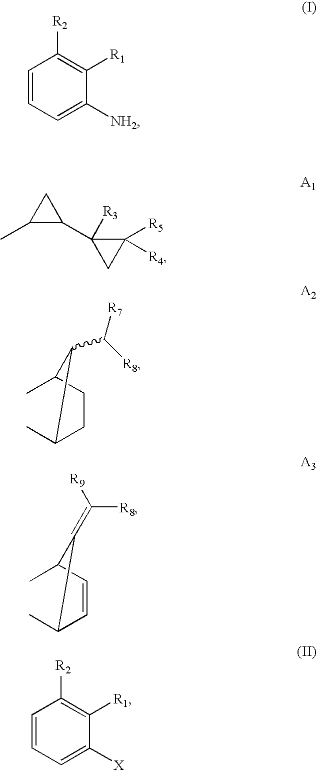

The present invention relates to a process for the amination of ortho-bicyclopropyl- or ortho-C₆-C₇alkyl-substituted halobenzenes, 5-halo-benzonorbornenes or 5-halo-benzonorbornadienes.

Ortho-bicyclopropyl- or ortho-C₆-C₇alkyl-substituted primary anilines such as, for example, 2-bicyclopropyl-2-yl-phenylamine and 2-(1,3-dimethyl-butyl)-phenylamine are valuable intermediates for the preparation of fungicides such as those described, for example, in WO 03/074491 and WO 03/010149.

5-amino-benzonorbornenes and 5-amino-benzonorbornadienes such as, for example, 9-isopropyl-1,2,3,4-tetrahydro-1,4-methano-naphthalen-5-ylamine, are valuable intermediates for the preparation of fungicides such as those described, for example, in WO 04/035589.

Agrochemicals are generally produced in large quantities. For example the fungicide chlorothalonil has been produced in the year 2005 in a quantity of over 23,000 metric tons.

In general terms, anilines with sterically less demanding ortho-substituents, such as the ortho-tolyl-amine, can be prepared from the reactions of the halobenzenes with ammonia by means of palladium-catalysed cross-coupling as described in Journal of the American Chemical Society, 128, 10028-10029, 2006. But the successful use of palladium-containing catalysts in a one-step amination of more sterically hindered halobenzenes, such as ortho-bicyclopropyl-substituted halobenzenes, 5-halo-benzonorbornenes or 5-halo-benzonorbornadienes, has not been described.

According to WO 03/074491, ortho-bicyclopropyl-substituted primary anilines can be prepared by reacting the corresponding ortho-bicyclopropyl-substituted halobenzenes in a two-step reaction first with benzophenone-imine in a palladium(II)-catalysed reaction and then reacting the reaction products with hydroxylamine hydrochloride and sodium acetate or with acids, for example hydrochloric acid. Such a reaction procedure for the preparation of primary anilines is unsuitable for the large-scale production of ortho-bicyclopropyl-substituted primary anilines, however, on account of the need for a second process step and the relative high cost of the benzophenoneimine. Furthermore, the reaction procedure is described in WO 03/074491 exclusively for bromo- or iodo-benzenes, not for chlorobenzenes. It has been found that the reaction procedure described in WO 03/074491 is poorly suited to the imination of the less reactive but more economically priced 2-(2-chlorophenyl)-bicyclopropanes in high yields.

The successful one-step-amination of the sterically hindered ortho-bicyclopropyl-substituted halobenzenes using copper-containing catalysts is known and is described in WO 06/061226. Such a reaction procedure for the preparation of primary anilines is not attractive for the large-scale production of ortho-alkyl-substituted primary anilines due to the high cost for the copper-salt waste management. Furthermore, it has been found that the reaction procedure described in WO 06/061226 is poorly suited for an amination of the less reactive but more economically priced 2-(2-chlorophenyl)bicyclopropanes in high yields.

Various 5-amino-benzonorbornenes or 5-amino-benzonorbornadienes, methods for their preparation and their use as intermediates in the production of microbiocides are described in WO 04/035589. According to WO 04/035589, these amines may be prepared as outlined in Scheme 1 below.

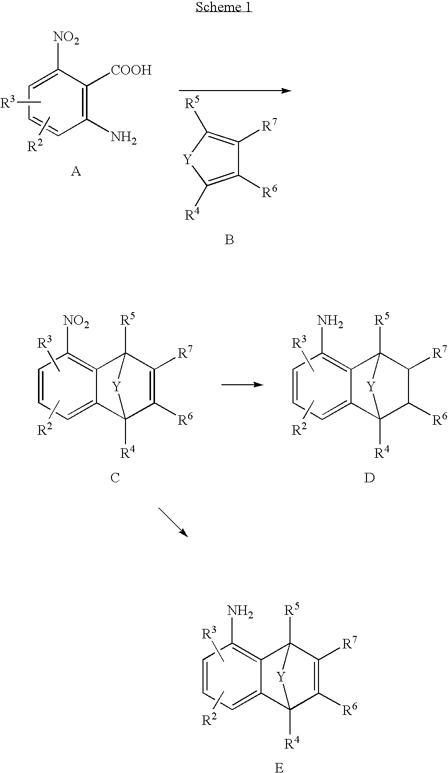

In the synthesis shown in Scheme 1, a 3-nitrobenzyne, generated from a 6-nitro-anthranilic acid (A), is reacted with a cyclic 1,4-diene (B), such as 5-isopropyl-cyclopentadiene, to form a 5-nitro-benzonorbornadiene (C) in a Diels-Alder reaction. Under standard catalytic reduction conditions (for example, using Raney nickel or palladium on carbon in a solvent such as methanol), both the 5-nitro group and the 2,3-double bond of the 5-nitro-benzonorbornadiene (C) are reduced to form the 5-amino-benzonorbornene (D). Under mild catalytic reduction conditions (for example, using metallic zinc in the presence of ammonium chloride or aluminium amalgam), the amino-benzonorbornadienes (E) are formed. An example of (D) is 5-amino-9-isopropyl-benzonorbornene, which is a precursor of an amide of, for example, 3-difluoromethyl-1-methyl-1H-pyrazole-4-carboxylic acid.

The problem with the synthesis outlined in Scheme 1 is that a number of unwanted isomeric impurities are formed. For example, in the preparation of the 5-nitro-benzonorbornadiene (C), where R⁴, R⁵, R⁶ and R⁷ are all H and Y is CH-iso-propyl, by the Diels-Alder reaction, the following regio-isomers are formed:

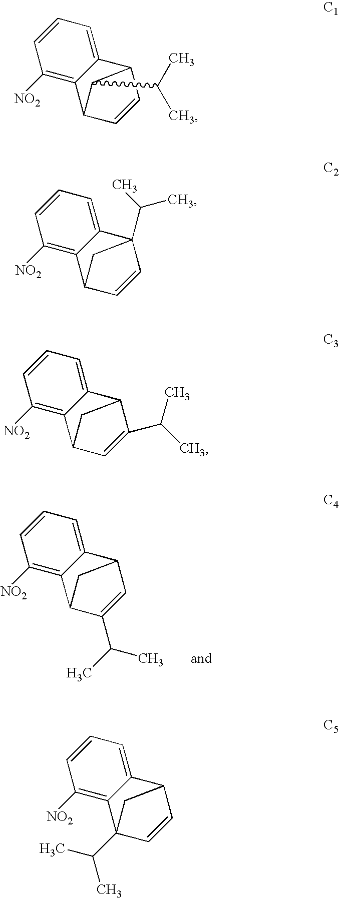

Unfortunately, the desired isomer C₁ is formed in relatively low yield. While the unwanted isomers may be removed, either at the end of the Diels-Alder reaction or at a later stage, by conventional techniques such as fractional crystallisation or fractional distillation or by chromatographic methods, this synthetic route is not well suited to large scale production.

The problem of the present invention is accordingly to provide a new process for the preparation of ortho-bicyclopropyl- or ortho-C₆-C₇alkyl-substituted primary anilines, 5-amino-benzonorbornenes and 5-amino-benzonorbornadienes, which avoids the above-mentioned disadvantages of the known process and makes it possible to prepare these compounds at economically reasonable cost and in easily manageable manner in high yields and good quality.

The present invention accordingly relates to a process for the preparation of compounds of formula I

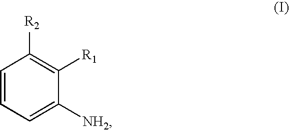

wherein
R₁ is 1,3-dimethyl-butyl, 1,3,3-trimethyl-butyl or a group A,

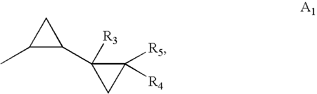

wherein R₃, R₄ and R₅ are each independently of the others hydrogen or C₁-C₄alkyl; and
R₂ is hydrogen; or
R₁ and R₂ together from the group A₂

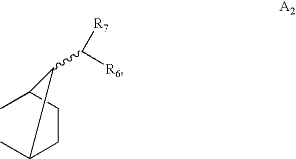

wherein R₈ and R₇ are each independently of the other hydrogen or C₁-C₄alkyl; or
R₁ and R₂ together from the group A₃

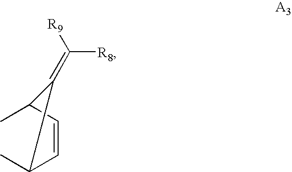

wherein R₈ and R₉ are each independently of the other hydrogen or C₁-C₄alkyl;
wherein a compound of formula II

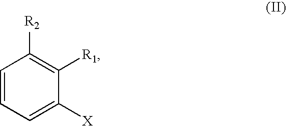

wherein R₁ and R₂ are as defined for formula I and X is bromine or chlorine, is reacted with ammonia in the presence of a base and a catalytic amount of at least one palladium complex compound, wherein the palladium complex compound comprises at least one ferrocenyl-biphosphine ligand.

Compounds of formula I occur in various stereoisomeric forms. The process according to the invention includes the preparation of said individual stereoisomeric forms and the preparation of mixtures of said stereoisomeric forms in any ratio.

The process according to the invention is suitable preferably for the preparation of compounds of formula I, wherein R₁ is a group A₁, wherein R₃, R₄ and R₅ are each independently of the others hydrogen or C₁-C₄alkyl; and R₂ is hydrogen; or R₁ and R₂ together from the group A₂, wherein R₆ and R₇ are each independently of the other hydrogen or C₁-C₄alkyl; or R₁ and R₂ together from the group A₃, wherein R₈ and R₉ are each independently of the other hydrogen or C₁-C₄alkyl.

The process according to the invention is suitable preferably for the preparation of compounds of formula I, wherein R₁ is A₁, R₃ is hydrogen or C₁-C₄alkyl and R₂, R₄ and R₅ are hydrogen.

The process according to the invention is suitable preferably for the preparation of compounds of formula I, wherein R₁ is A₁, R₃ is hydrogen or methyl and R₂, R₄ and R₅ are hydrogen.

The process according to the invention is suitable especially for the preparation of compounds of formula IA

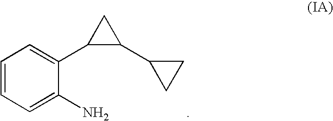

The process according to the invention is suitable preferably for the preparation of compounds of formula I, wherein R₁ is 1,3-dimethyl-butyl and R₂ is hydrogen.

The process according to the invention is suitable preferably for the preparation of compounds of formula I, wherein R₁ is 1,3,3-trimethyl-butyl and R₂ is hydrogen.

The process according to the invention is suitable preferably for the preparation of compounds of formula I wherein R₁ and R₂ together form the group A₂, wherein R₆ and R₇ are each independently of the other hydrogen or C₁-C₄alkyl.

The process according to the invention is suitable preferably for the preparation of compounds of formula I wherein R₁ and R₂ together from the group A₂, wherein R₆ and R₇ are each methyl.

The process according to the invention is suitable preferably for the preparation of compounds of formula I wherein R₁ and R₂ together from the group A₃, wherein R₈ and R₉ are each independently of the other hydrogen or C₁-C₄alkyl.

The process according to the invention is suitable preferably for the preparation of compounds of formula I wherein R₁ and R₂ together from the group A₃, wherein R₈ and R₉ are each methyl.

Compounds of formula II wherein X is bromine are preferably used in the process according to the invention.

Compounds of formula II wherein X is chlorine are preferably used in the process according to the invention.

In the process according to the invention compounds of formula II can be used typically in concentrations of between 0.01 M and 5 M. More preferably, compounds of formula II are used in concentrations of between 0.1 M and 5 M. Even more preferably, compounds of formula I are used in concentrations of between 0.1 M and 2 M. The possibility of using high concentrations of compounds of formula II is an important advantage of the process according to the invention as with high concentrations of educts less solvent is needed, which makes the process according to the invention especially suitable for large-scale production.

The palladium complex compounds which are used in the process according to the invention are formed from a palladium precursor and at least one ferrocenyl-biphosphine ligand. In the process according to the invention, the palladium complex compounds are preferably present in dissolved form as palladium-ligand complexes.

The palladium complex compounds may be used as already formed palladium complex compounds in the process according to the invention or are formed in situ in the process according to the invention.

In order to form palladium complex compounds, a palladium precursor is reacted with at least one ferrocenyl-biphosphine ligand. In the event of incomplete reaction, it can be the case that minor amounts of palladium precursor or of ligand do not dissolve in the reaction mixture.

Suitable palladium precursors are palladium acetate, palladium dichloride, palladium dichloride solution, palladium₂ (dibenzylidene-acetone)₃ or palladium (dibenzylidene-acetone)₂, palladium tetrakis(triphenylphosphine), palladium-on-carbon, palladium dichlorobis(benzonitrile), palladium (tris-tert-butylphosphine)₂ or a mixture of palladium₂ (dibenzylidene-acetone)₃ and palladium (tris-tentbutylphosphine)₂.

Ferrocenyl-biphosphine ligands are bidentate tertiary phosphine ligands commonly used in palladium-catalyzed reactions. Such bidentate ligands occupy two coordination sites and hence are able to chelate the palladium species.

Suitable ferrocenyl-biphosphine ligands are:

(R)-(+14(S)-2-(dicyclohexylphosphino)ferrocenyl]ethyldi-tert-butylphosphine

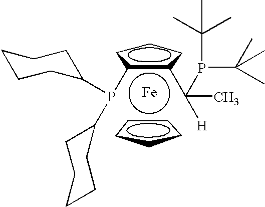

1,1′-bis(diphenylphosphino)ferrocene (dppf), 1,1′-bis(di-tert-butylphosphino)-ferrocene, (R)-(−)-1-[(S)-2-(bis(4-trifluoromethylphenyl)phosphino)ferrocenyl]ethyl-di-tert-butylphosphine, (R)-(+14(S)-2-(di(3,5-bis-trifluoromethylphenyl)phosphino)ferrocenyl]ethyldicyclohexyl-phosphine, (R)-(−)-1-[(S)-2-(di(3,5-bis-trifluoromethylphenyl)phosphino)ferrocenyl]ethyldi(3,5-dimethylphenyl)phosphine, (R)-(−)-1-[(S)-2-(dicyclohexylphosphino)ferrocenyl]-ethyldicyclohexylphosphine, (S)-(+)-1[(R)-2-(dicyclohexylphosphino)ferrocenyl]ethyldicyclo-hexylphosphine, (S)-(+)-1-[(R)-2-(dicyclohexylphosphino)ferrocenyl]ethyldiphenylphosphine, (R)-(−)-1-[(S)-2-(bis(3,5-dimethyl-4-methoxyphenyl)phosphino)ferrocenyl]ethyldicyclohexyl-phosphine, (S)-(+)-1-[(R)-2-(di-furylphosphino)ferrocenyl]ethyldi-3,5-xylylphosphine, (R)-(−)-1-[(S)-2-(diphenylphosphino)ferrocenyl]ethyldi-tert-butylphosphine, (S)-(+)-1-[(R)-2-(diphenylphosphino)ferrocenyl]ethyldi-tert-butylphosphine, (R)-(−)-1-[(S)-2-(diphenylphosphino)ferrocenyl]ethyldicyclohexylphosphine, (R)-(+)-1-[(R)-2-(diphenyl-phosphino)ferrocenyl]ethyldicyclohexylphosphine, (S)-(+)-1-[(R)-2-(diphenylphosphino)-ferrocenyl]ethyldicyclohexylphosphine, (R)-(−)-1-[(S)-2-(dicyclohexylphosphino)ferrocenyl]-ethyldiphenylphosphine, (R)-(−)-1-[(S)-2-(diphenyl)phosphino)ferrocenyl]ethyldi(3,5-dimethyl-phenyl)phosphine, (R)-(−)-1-[(S)-2-(di-tert-butyl-phosphino)ferrocenyl]ethyl-di-o-tolylphosphine

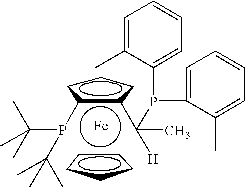

(R)-(−)-1-[(S)-2-(bis(3,5-dimethyl-4-methoxyphenyl)phosphino)ferrocenyl]ethyl-di-tert-butylphosphine

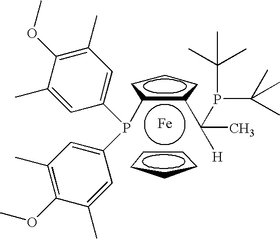

(R)-(−)-1-[(S)-2-(diethylphosphino)ferrocenyl]-ethyl-di-tert-butylphosphine

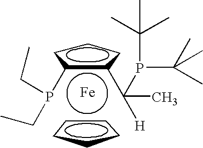

(R)-(−)-1-[(S)-2-(P-methyl-P-isopropyl-phosphino)ferrocenyl]ethyldicyclohexylphosphine

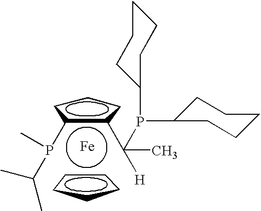

(R)-(−)-1-[(S)-2-(P-methyl-P-phenyl-phosphino)ferrocenyl]ethyl-di-tert-butylphosphine

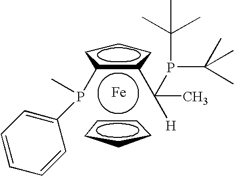

and racemic mixtures thereof, especially racemic mixtures of 1-[2-(di-tert-butylphosphino)-ferrocenyl]ethyl-di-o-tolylphosphine, 1-[2-(dicyclohexylphosphino)ferrocenyl]ethyldi-tert-butyl-phosphine and 1-[2-(diphenylphosphino)ferrocenyl]ethyldicyclohexylphosphine.

One palladium complex compound or a mixture of palladium complex compounds may be used in the process according to the invention.

For formation of the palladium complex compound, preference is given to the use, as palladium precursor, of palladium acetate, palladium₂ (dibenzylidene-acetone)₃, palladium (dibenzylidene-acetone)₂, palladium dichloride solution, palladium dichloride or a mixture of palladium₂ (dibenzylidene-acetone)₃ and palladium (tris-tert-butylphosphine)₂. Special preference is given to the use of palladium acetate or palladium dichloride.

At least one ligand is used for formation of the palladium complex compound.

Preference is given to the use of palladium complex compounds which comprise at least one ligand selected from (R)-(−)-1-(S)-2-(dicyclohexylphosphino)ferrocenyl]ethyldi-tert-butyl-phosphine and racemic 1-[2-(dicyclohexylphosphino)ferrocenyl]ethyldi-tert-butylphosphine.

Preference is given to the use of palladium complex compounds which comprise racemic 1-[2-(dicyclohexylphosphino)ferrocenyl]ethyldi-tert-butylphosphine.

Palladium complex compounds, palladium precursors and/or ligands are used in catalytic amounts in the process according to the invention.

Palladium complex compounds are used preferably in a ratio of from 1:10 to 1:10 000 relative to compounds of formula II, especially in a ratio of from 1:100 to 1:1000.

Palladium precursors are used preferably in a ratio of from 1:10 to 1:10 000 relative to compounds of formula II, especially in a ratio of from 1:100 to 1:1000.

Ligands are used preferably in a ratio of from 1:10 to 1:10 000 relative to compounds of formula II, especially in a ratio of from 1:100 to 1:1000.

Suitable bases are, for example, alcoholates, e.g. sodium tert-butanolate, potassium tert-butanolate, sodium methanolate or sodium ethanolate, or inorganic bases such as carbonates, e.g. K₂CO₃, Na₂CO₃ or Cs₂CO₃, hydroxides, e.g. NaOH or KOH, phosphates, e.g. K₃PO₄, or amides, e.g. LiNH₂, NaNH₂ or KNH₂; in one embodiment, preference is given to alcoholates and special preference is given to sodium tert-butanolate; in another embodiment, preference is given to amides and special preference is given to NaNH₂, KNH₂ or a mixture thereof.

When NaOH or KOH is used as the base, a phase transfer catalyst such as, for example, cetyltrimethylammonium bromide may be used.

Suitable amounts of base for this reaction are, for example, from 1 to 3 equivalents, especially from 1 to 2 equivalents.

The reaction according to the invention may be carried out in an inert solvent.

In one embodiment of the invention, the reaction according to the invention is carried out in an inert solvent. Suitable solvents are, for example, a compound of formula V

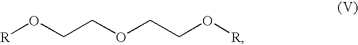

wherein R is C₁-C₆alkyl, preferably methyl; dimethoxyethane; tert-butyl methyl ether; tetrahydrofuran; dioxane; tert-butanol; toluene; xylene; anisol or trimethylbenzenes such as, for example, mesitylene; and also mixtures thereof; preferred solvents are dimethoxyethane, tetrahydrofuran or diglyme.

In that embodiment, the inert solvent is preferably anhydrous.

The reaction according to the invention is carried out at ambient temperature or at elevated temperature, preferably in a temperature range from 50° C. to 180° C., especially in a temperature range from 50° C. to 120° C.

The reaction according to the invention is typically carried out at elevated pressure. In one embodiment, the reaction according to the invention is carried at a pressure of between 1-100 bar, preferably between 5-80 bar.

The reaction time of the reaction according to the invention is generally from 1 to 48 hours, preferably from 4 to 30 hours, especially from 4 to 18 hours.

The reaction according to the invention may be carried out in an inert gas atmosphere. For example, nitrogen or argon is used as inert gas.

In one embodiment of the reaction according to the invention, the reaction is carried out in a nitrogen atmosphere.

In the reactions according to the invention, ammonia is used in equimolar amounts or in excess relative to compounds of formula II, preferably in an up to 500-fold excess, especially in an up to 200-fold excess, more especially in an 80-fold to 120-fold excess. In one embodiment of the invention, ammonia is used with a 10-fold to 30-fold excess.

In the process according to the invention, ammonia can be introduced into the reaction vessel in liquid form or in gaseous form.

The compounds of formula II wherein X is bromine, R₁ is a group A₁ and R₂ is hydrogen are generally known and can be prepared according to the processes described in WO 03/074491. The compounds of formula II wherein X is chlorine, R₁ is a group A₁ and R₂ is hydrogen can be prepared in analogous manner to the processes described in WO 03/074491 for the corresponding compounds of formula II wherein X is bromine, R₁ is a group A₁ and R₂ is hydrogen. For example, the compound of formula II wherein X is chlorine, R₁ is a group A₁ and R₂, R₃, R₄ and R₅ is hydrogen (compound no. B1) can be prepared as shown in Reaction Scheme 1 and as explained by Examples A1-A3 which follow:

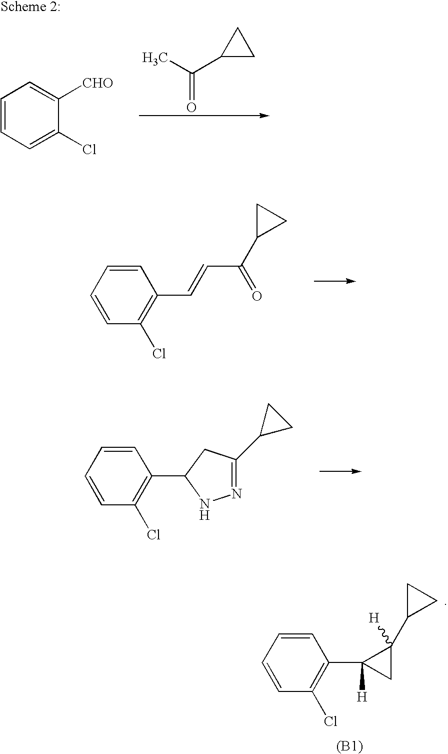

### Preparation Example A1

### Preparation of 3-(2-chlorophenyl)-1-cyclopropyl-propenone

67 g of 30% sodium hydroxide solution are mixed with 350 ml of water and 97.5 g (1.1 mol) of cyclopropyl methyl ketone and heated to 90° C., with stirring. 143.5 g (1 mol) of 2-chloro-benzaldehyde are added dropwise to the resulting mixture and stirring is carried out for 5 hours. During stirring, after 2 hours and after a further 3 hours, 2 ml of cyclopropyl methyl ketone are added on each occasion. After a total reaction time of 6 hours, cooling to 50° C. is carried out. The reaction mixture is filtered and the phases are separated. The organic phase is concentrated. 188.6 g of 3-(2-chlorophenyl)-1-cyclopropyl-propenone are obtained in the form of a yellow oil.

¹H NMR (CDCl₃): 0.95-1.04 (m, 2H); 1.16-1.23 (m, 2H); 2.29-2.37 (m, 1H); 6.83 (d, J=15 Hz); 7.27-7.35 (m, 2H); 7.40-7.47 (m, 1H); 8.03 (d, J=15 Hz)

### Preparation Example A2

### Preparation of 5-(2-chlorophenyl)-3-cyclopropyl-4,5-dihydro-1H-pyrazole

250 g of ethanol are added to 188.6 g of the 3-(2-chlorophenyl)-1-cyclopropyl-propenone (1 mol) prepared according to A1. 53 g (1.05 mol) of hydrazine hydrate are added dropwise at 20° C., with stirring. The reaction mixture is stirred at 70° C. for 2 hours. The reaction mixture is then cooled to 50° C. A mixture of 5.5 g of oxalic acid dihydrate (0.044 mol) and 20 g of ethanol is added, whereupon a solid precipitates out. The reaction mixture is cooled to 25° C. and is filtered through a sintered-glass suction filter and washed with 50 g of ethanol. A yellow filtrate is obtained, which is concentrated by evaporation using a rotary evaporator at 60° C. and down to 20 mbar to form a yellow oil. 201.5 g of an isomeric mixture having the main component 5-(2-chlorophenyl)-3-cyclopropyl-4,5-dihydro-1H-pyrazole are obtained in the form of a yellow oil.

### Preparation Example A3

### Synthesis of 2-(2-chlorophenyl)bicyclopropyl

To a solution of 50 g (0.36 mol) of potassium carbonate in 600 g of ethylene glycol there are added at 190° C., in the course of 2 hours, 201.5 g of 5-(2-chlorophenyl)-3-cyclopropyl-4,5-dihydro-1H-pyrazole, prepared as described under A2. Stirring is then carried out for 2 hours at 190° C. The end of the reaction is indicated by cessation of the evolution of gas. The reaction mixture is then cooled to 100° C., whereupon phase separation occurs and the upper, product phase is separated off. 158 g of 2-(2-chlorophenyl)bicyclopropyl are obtained as crude product, which may be further purified, for example by distillation.

¹H NMR (CDCl₃): 0.0-1.13 (m, 8H); 1.95-2.02 (m, 0.63H, trans isomer) and 2.14-2.22 (m, 0.37H, cis isomer); 6.88-6.94 (m); 7.05-7.24 (m); 7.31-7.42 (m)

The compounds of formula II wherein X is bromine or chlorine and R₁ and R₂ together form a group A₂ or A₃ can be prepared according to the processes as described in WO 07/068,417.

Palladium complex compounds, palladium precursors and ligands as used in the process according to the invention are generally known and, for the most part, commercially available.

The present invention will be explained in greater detail using the following Examples:

### EXAMPLE P1

### Preparation of 2-biscyclopropylaniline (substrate/catalyst-ratio=20:1)

A mixture of 385 mg 2-(2-chlorophenyl)bicyclopropyl (2 mmol, trans/cis ratio ca. 3:2), 288 mg sodium tert. butoxide (3 mmol), 22.4 mg palladium acetate (0.1 mmol), 61 mg R(−)-di-tert.butyl-[1-[(S)-2-(dicyclohexylphosphanyl)-1-ferrocenyl]ethyl]phosphine (0.11 mmol), 4 g ammonia gas (0.235 mol) and 1.5 ml diglyme was stirred at elevated pressure in a pressure vessel at 160° C. for 18 h (argon atmosphere). Then the mixture was diluted with 20 ml of ethylacetate and filtered. The remaining liquid phase was concentrated under reduced pressure and the crude material purified by column chromatography over silicagel (eluent:ethylacetate/heptane 1:5). 0.26 g (75% of theory) of pure 2-biscylopropylaniline were obtained as a slightly brownish liquid (trans/cis ratio ca. 1:1).

### EXAMPLE P2

### Preparation of 2-biscyclopropylaniline (substrate/catalyst-ratio=100:1)

A mixture of 385 mg 2-(2-chlorophenyl)bicyclopropyl (2 mmol, trans/cis ratio ca. 3:2), 288 mg sodium tert. butoxide (3 mmol), 4.5 mg palladium acetate (0.02 mmol), 12.2 mg R(−)-di-tert.butyl-[1-[(S)-2-(dicyclohexylphosphanyl)-1-ferrocenyl]ethyl]phosphine (0.022 mmol), 4 g ammonia gas (0.235 mol) and 1.5 ml tetrahydrofurane was stirred at elevated pressure in a pressure vessel at 120° C. for 17 h (argon atmosphere). The yield of 2-biscylopropylaniline was determined by gaschromatography: 86% (sum of isomers).

### EXAMPLE P3

### Preparation of 2-biscyclopropylaniline (substrate/catalyst-ratio=100:1)

A mixture of 385 mg 2-(2-chlorophenyl)bicyclopropyl (2 mmol, trans/cis ratio ca. 3:2), 288 mg sodium tert. butoxide (3 mmol), 4.5 mg palladium acetate (0.02 mmol), 12.2 mg R(−)-di-tert.butyl-[1-[(S)-2-(dicyclohexylphosphanyl)-1-ferrocenyl]ethyl]phosphine (0.022 mmol), 4 g ammonia gas (0.235 mol) and 1.5 ml dimethoyethane was stirred at elevated pressure in a pressure vessel at 120° C. for 16 h (argon atmosphere). The yield of 2-biscylopropylaniline was determined by gaschromatography: 80% (sum of isomers).

### EXAMPLE P3

### Preparation of 2-biscyclopropylaniline (substrate/catalyst-ratio=500:1)

A mixture of 385 mg 2-(2-chlorophenyl)bicyclopropyl (2 mmol, trans/cis ratio ca. 3:2), 288 mg sodium tert. butoxide (3 mmol), 0.9 mg palladium acetate (0.004 mmol), 2.44 mg R-(−)-di-tert.butyl-[1-[(S)-2-(dicyclohexylphosphanyl)-1-ferrocenyl]ethyl]phosphine (0.0044 mmol), 4 g ammonia gas (0.235 mol) and 1.5 ml dimethoxyethane was stirred at elevated pressure in a pressure vessel at 120° C. for 16 h (argon atmosphere). The yield of 2-biscylopropylaniline was determined by gaschromatography: 86% (sum of isomers).

### EXAMPLE P4

### Preparation of 2-biscyclopropylaniline (substrate/catalyst-ratio=100:1)

In an argon atmosphere 599 mg (1.1 mmol) (R)-(−)-1-[(S)-2-(dicyclohexylphosphino)-ferrocenyl]ethyldi-tert-butylphosphine and 160 mg (0.24 mmol) palladium acetate (trimer) in 2 ml dimethoxyethane are stirred for 30 min at room temperature and 1 min at 50° C. In an argon atmosphere the catalytic system and 2 ml dimethoxyethane are added to 20.8 g (95%, 0.11 mol) 2-(2-chlorophenyl)bicyclopropyl and 10.5 g (0.11 mol) sodium tert-butanolate in 30 ml dimethoxyethane in a autoclave. Subsequently 36 g (2.11 mol) ammonia (liquid) are added and the suspension is heated to 119° C. giving a pressure of 61 bar. After 18 h the reaction mass is cooled to room temperature, flushed twice with nitrogen and quenched with 30 ml of water. The reaction mass is filtered via hyflow, the filter is rinsed with xylene and water and the aqueous phase is extracted three times with xylene. The organic solvents are removed in vacuo. The content of 2-biscyclopropylaniline was determined by gas chromatography: 78% (area GC) leaving 4.97% (area GC) of starting material. Additionally 3.57% (area GC) of a dimeric by-product and 3.55% (area GC) dehalogenated by-product are detected.

### EXAMPLE P5

### Preparation of 5-amino-9-isopropylbenzonorbornene-syn enriched (substrate/catalyst-ratio=100:1)

A mixture of 221 mg 5-chloro-9-isopropylbenzonorbornene (1 mmol, >98% syn isomer), 192 mg sodium tert. butoxide (2 mmol), 2.25 mg palladium acetate (0.01 mmol), 6.1 mg R(−)-di-tert.butyl-[1-[(S)-2-(dicyclohexylphosphanyl)-1-ferrocenyl]ethyl]phosphine (0.011 mmol), 4 g ammonia gas (0.235 mol) and 5 ml dimethoxyethane was stirred at elevated pressure in a pressure vessel at 100° C. for 21 h (argon atmosphere). The yield of 5-amino-9-isopropylbenzonorbornene was determined by gaschromatography: 90% (>98% syn isomer).

Using the above Examples, the following compounds of formula I can be prepared:

TABLE 1
Compounds of formula I

| | | | | | | | | | (I) |
|---|---|---|---|---|---|---|---|---|---|
| 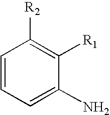 | | | | | | | | | |
| Comp. | | | | | | | | | |
| no. | R₁ | R₂ | R₃ | R₄ | R₅ | R₆ | R₇ | R₈ | R₉ |
| A1 | A₁ | H | H | H | H | — | — | — | — |
| A2 | A₁ | H | CH₃ | H | H | — | — | — | — |

| | | | | | | | | |
|---|---|---|---|---|---|---|---|---|
| A3 | A₂ | — | — | — | CH₃ | CH₃ | — | — |
| A4 | A₂ | — | — | — | H | H | — | — |
| A5 | A₃ | — | — | — | — | — | CH₃ | CH₃ |
| A6 | A₃ | — | — | — | — | — | H | H |

The following compounds of formula II are suitable for use in the process according to the invention:

TABLE 2
Compounds of formula II

| | | | | | | | | | | (II) |
|---|---|---|---|---|---|---|---|---|---|---|
| 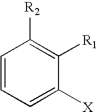 | | | | | | | | | | |
| Comp. | | | | | | | | | | |
| no. | X | R₁ | R₂ | R₃ | R₄ | R₅ | R₆ | R₇ | R₈ | R₉ |
| B1 | Cl | A₁ | H | H | H | H | — | — | — | — |
| B2 | Cl | A₁ | H | CH₃ | H | H | — | — | — | — |
| B3 | Cl | A₂ | | — | — | — | CH₃ | CH₃ | — | — |
| B4 | Cl | A₂ | | — | — | — | H | H | — | — |
| B5 | Cl | A₃ | | — | — | — | — | — | CH₃ | CH₃ |
| B6 | Cl | A₃ | | — | — | — | — | — | H | H |
| B7 | Br | A₁ | H | H | H | H | — | — | — | — |
| B8 | Br | A₁ | H | CH₃ | H | H | — | — | — | — |

| | | | | | | | | | |
|---|---|---|---|---|---|---|---|---|---|
| B9 | Br | A₂ | — | — | — | CH₃ | CH₃ | — | — |
| B10 | Br | A₂ | — | — | — | H | H | — | — |
| B11 | Br | A₃ | — | — | — | — | — | CH₃ | CH₃ |
| B12 | Br | A₃ | — | — | — | — | — | H | H |

As a result of the provision of the present invention, it is possible to animate ortho-bicyclopropyl-substituted halobenzenes, 5-halo-benzonorbornenes and 5-halo-benzonorbornadienes in high yields and with little outlay.

The starting compounds of the process of the present invention are distinguished by being readily accessible and easily handled and, in addition, they are economically priced.

In a preferred embodiment of the process according to the invention, the palladium and/or the palladium complex compound used in the process is recycled. This embodiment constitutes a variant of the process according to the invention which is especially interesting from an economic point of view.

In a preferred embodiment of the invention, compounds of formula II wherein X is chlorine are used. The starting compounds of this preferred embodiment of the process of the invention are distinguished by being especially readily accessible and economical. It is known, however, that, under the conditions of palladium-catalysed cross-coupling, this class of starting compounds, the sterically hindered, deactivated, at least ortho-substituted chlorobenzene substrates, are especially difficult to animate because of the extremely low reactivity of the chlorine leaving group, compared to bromobenzene substrates. As this embodiment of the invention makes those starting compounds accessible to the palladium-catalysed cross-coupling, this embodiment accordingly constitutes a variant of the process according to the invention, which is especially interesting from an economic point of view.

What is claimed is:

1. A process for the preparation of compounds of formula I 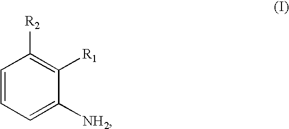
wherein
R₁ is 1,3-dimethyl-butyl, 1,3,3-trimethyl-butyl or a group A₁ 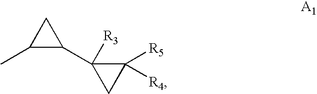
wherein R₃, R₄ and R₅ are each independently of the others hydrogen or C₁-C₄alkyl; and
R₂ is hydrogen; or
R₁ and R₂ together from the group A₂ 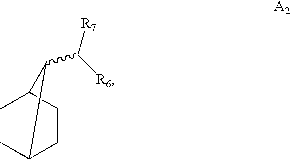
wherein R₈ and R₇ are each independently of the other hydrogen or C₁-C₄alkyl; or
R₁ and R₂ together from the group A₃ 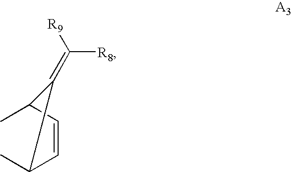
wherein R₈ and R₉ are each independently of the other hydrogen or C₁-C₄alkyl;
wherein a compound of formula II 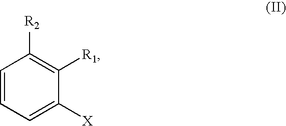
wherein R₁ and R₂ are as defined for formula I and X is bromine or chlorine, is reacted with ammonia in the presence of a base and a catalytic amount of at least one palladium complex compound, wherein the palladium complex compound comprises at least one ferrocenyl-biphosphine ligand.

2. A process according to claim 1, wherein R₁ is a group A₁, wherein R₃, R₄ and R₅ are each independently of the others hydrogen or C₁-C₄alkyl; and R₂ is hydrogen; or R₁ and R₂ together from the group A₂, wherein R₈ and R₇ are each independently of the other hydrogen or C₁-C₄alkyl; or R₁ and R₂ together from the group A₃, wherein R₈ and R₉ are each independently of the other hydrogen or C₁-C₄alkyl.

3. A process according to claim 1, wherein the palladium complex compound comprises at least one ligand selected from R(−)-di-tert-butyl-[1-[(S)-2-(dicyclohexylphosphinyl)ferrocenyl]ethyl]phosphine and racemic di-tert-butyl-[1-[2-(dicyclohexylphosphinyl)ferrocenyl]ethyl]phosphine.

4. A process according to claim 1, wherein the palladium complex compound comprises racemic di-tert-butyl-[1-[2-(dicyclohexylphosphinyl)ferrocenyl]ethyl]phosphine.

5. A process according to claim 1, wherein the palladium complex compound is used in a ratio of from 1:10 000 to 1:10 relative to the compound of formula II.

6. A process according to claim 1, wherein the compound of formula II is used in a concentration of between 0.01 M and 5 M.

7. A process according to claim 1, wherein X is chlorine.
